(19) United States
(12) Patent Application Publication
(10) Pub. No.: US 2015/0045470 A1
(43) Pub. Date: Feb. 12, 2015

(54) ANTIBODY PURIFICATION METHOD, AND CARRIER FOR USE IN PURIFICATION OF ANTIBODY

(71) Applicant: JSR CORPORATION, Minato-ku, Tokyo (JP)

(72) Inventor: Satoshi Katayose, Minato-ku (JP)

(73) Assignee: JSR CORPORATION, Minato-ku, Tokyo (JP)

(21) Appl. No.: 14/383,322

(22) PCT Filed: Mar. 5, 2013
(86) PCT No.: PCT/JP2013/055943

§ 371 Date: Sep. 5, 2014

(30) Foreign Application Priority Data
Mar. 6, 2012 (JP) 2012-048863

(51) Int. Cl. C07K 1/22; B01J 20/28; B01J 20/26; C07K 16/00

(52) U.S. Cl. CPC C07K 1/22; C07K 16/00; B01J 20/28016; B01J 20/265; B01J 20/28095
USPC 521/149; 530/387.1; 525/327.3; 525/332.2; 524/576

(57) ABSTRACT

An antibody purification method includes: bringing a solution that includes an antibody and has a pH of 3.0 or more and less than 5.6 into contact with a carrier that includes a 2,3-dihydroxypropyl group to adsorb the antibody on the carrier; and bringing the carrier that has been brought into contact with the solution, into contact with an eluent having a pH of 5.6 or more and less than 10 to elute the antibody from the carrier.

### TECHNICAL FIELD

The present invention relates to an antibody purification method and an antibody purification carrier.

### BACKGROUND ART

Various methods have been used to purify an antibody from an antibody-containing sample taking account of the desired purity and the intended use. An ammonium sulfate precipitation method (fractional precipitation method) that utilizes ammonium sulfate as a precipitant has been widely used when the degree of purification may be relatively low.

The ammonium sulfate precipitation method utilizes a protein salting-out phenomenon, and precipitates the antibody, or collects a supernatant liquid including the antibody utilizing the difference in salting-out ammonium sulfate concentration between the antibody and other proteins that are present together with the antibody. The ammonium sulfate precipitation method can process a large amount of antibody at low cost. However, troublesome procedures are required to determine the ammonium sulfate concentration for separating the antibody. Since the resulting antibody includes a large amount of ammonium sulfate, a desalting treatment such as dialysis is normally necessary. Moreover, since a large amount of antibody is lost due to the desalting treatment, it is difficult to apply the ammonium sulfate precipitation method to a small amount of antibody. Therefore, when purifying an antibody using the ammonium sulfate precipitation method, it is normally necessary to combine another purification method with the ammonium sulfate precipitation method in order to increase the purity of the resulting antibody.

Affinity chromatography that utilizes a carrier to which a protein that binds to an antibody molecule (e.g., protein A or protein G) is bound as a ligand has been widely used to purify an IgG-type antibody to have high purity (see JP-A-7-146280 and U.S. Pat. No. 6,399,750). When using affinity chromatography, an antibody-containing sample is brought into contact with the carrier at an almost neutral pH, and the antibody is eluted using an acidic eluent having a pH of about 2 to 3.

In order to prevent a situation in which the antibody is denatured due to the acidic eluent, it is necessary to immediately neutralize the acidic eluent including the collected antibody so that the eluent has an almost neutral pH. An antibody purified by affinity chromatography has high purity. However, precipitation may occur due to the acidic eluent, and the activity of the antibody may be impaired. The antibody may exhibit poor bindability to the carrier, and it may be difficult to purify the antibody depending on the antibody subclass and the animal species from which the antibody is derived. Moreover, since a carrier to which protein A or protein G is bound is expensive, the purification cost may increase.

An IgM-type antibody has low affinity to protein A and protein G, and it is difficult to appropriately purify an IgM-type antibody by affinity chromatography (see JP-A-2007-332080). Therefore, it is necessary to purify an IgM-type antibody by gel filtration chromatography, ion-exchange chromatography, or a combination thereof, and a convenient and practical purification method has been desired.

Since a known antibody purification method utilizes a solution having a high salt concentration or a strongly acidic eluent during the purification process, it is normally necessary to perform a desalting treatment and a neutralization treatment on the collected antibody. It is difficult to conveniently purify a small amount of antibody using the ammonium sulfate precipitation method. On the other hand, affinity chromatography has a drawback in that it is difficult to inexpensively purify a large amount of antibody to have relatively high purity. Moreover, the antibody may be irreversibly denatured when a strongly acidic eluent is used, and it is difficult to apply affinity chromatography depending on the antibody class/subclass.

### SUMMARY OF INVENTION

### Technical Problem

Several aspects of the invention provide an antibody purification method and an antibody purification carrier that can prevent a situation in which an antibody is denatured and inactivated during purification.

### Solution to Problem

The inventor of the invention found that an antibody is adsorbed on a carrier that includes a 2,3-dihydroxypropyl group as a ligand in a solution having a pH of 3.0 or more and less than 5.6, and found that an antibody can be purified by utilizing such a carrier.

According to one aspect of the invention, an antibody purification method includes bringing a solution that includes an antibody and has a pH of 3.0 or more and less than 5.6 into contact with a carrier that includes a 2,3-dihydroxypropyl group, and bringing the carrier that has been brought into contact with the solution, into contact with an eluent having a pH of 5.6 or more and less than 10.

In the antibody purification method, the antibody may be an immunoglobulin.

According to another aspect of the invention, an antibody purification carrier includes a 2,3-dihydroxypropyl group, the 2,3-dihydroxypropyl group being present on a surface of the antibody purification carrier.

The carrier may be a porous particle.

The carrier may be a particle that includes a magnetic material.

### Advantageous Effects of Invention

The antibody purification method can adsorb the antibody on a ligand and elute impurities in a solution having a pH of 3.0 or more and less than 5.6, and elute the antibody using a relatively neutral aqueous solution having a pH of 5.6 or more and less than 10. According to the antibody purification method, since it is unnecessary to perform a desalting treatment and a neutralization treatment that are required for a known antibody purification method, it is possible to prevent a situation in which the antibody is denatured and inactivated during purification.

Specifically, the antibody purification method is a novel and useful antibody purification method based on a principle that differs substantially completely from that of known antibody purification methods such as the ammonium sulfate precipitation method and affinity chromatography (see “BACKGROUND ART”).

### BRIEF DESCRIPTION OF DRAWINGS

FIG. 1 illustrates the sodium dodecyl sulfate-polyacrylamide gel electrophoresis (SDS-PAGE) assay results for a partially purified IgG solution, a solution obtained by an adsorption step, and a solution collected in an elution step.

FIG. 2 illustrates the SDS-PAGE assay results for a supernatant liquid of a dispersion collected in an adsorption step (Example 2).

FIG. 3 illustrates the SDS-PAGE assay results for a supernatant liquid of a dispersion collected in an adsorption step (Comparative Example 1).

FIG. 4 illustrates the SDS-PAGE assay results for a solution collected in an elution step (Example 3).

FIG. 5 illustrates the SDS-PAGE assay results for a solution collected in an elution step (Example 4 and Comparative Example 2).

### DESCRIPTION OF EMBODIMENTS

An antibody purification method and an antibody purification carrier according to several exemplary embodiments of the invention are described below.

### 1. ANTIBODY PURIFICATION CARRIER

An antibody purification carrier (hereinafter may be referred to as “carrier”) according to one embodiment of the invention includes a 2,3-dihydroxypropyl group (HO—CH₂—CH(OH)—CH₂—), the 2,3-dihydroxypropyl group being used as an antibody adsorption ligand.

The expression “the carrier includes a 2,3-dihydroxypropyl group” used herein means that a 2,3-dihydroxypropyl group is present at the terminal of the molecule that forms the carrier. Therefore, a case where the carrier includes a group having the terminal structure of a 2,3-dihydroxypropyl group, such as a 3,4-dihydroxybutyl group, a 4,5-dihydroxypentyl group, a 2,3-dihydroxypropyloxy group, or a 2,3-dihydroxypropylthio group, falls under the expression “the carrier includes a 2,3-dihydroxypropyl group”.

Examples of the carrier according to one embodiment of the invention include a porous particle, a particle that includes a magnetic material, a filtration filter, a filtration fiber, a chromatography column particle, a batch processing particle, and the like. The carrier according to one embodiment of the invention is preferably a porous particle or a particle that includes a magnetic material.

### 1.1. Structure

The carrier according to one embodiment of the invention preferably includes a 2,3-dihydroxypropyl group that is present on the surface thereof. For example, at least the surface of the carrier according to one embodiment of the invention may be formed of a polymer part, and at least the surface of the polymer part may include a 2,3-dihydroxypropyl group. In this case, the polymer part may be formed by copolymerizing a monomer part including a monomer that includes a 2,3-dihydroxypropyl group, or may be formed by copolymerizing a monomer part including a monomer that produces a 2,3-dihydroxypropyl group through hydrolysis, and hydrolyzing the resulting copolymer.

The entirety of the carrier according to one embodiment of the invention may be formed of the polymer part, or the carrier according to one embodiment of the invention may have a core-shell structure, and the shell may be formed of the polymer part.

The 2,3-dihydroxypropyl group included in the carrier according to one embodiment of the invention functions as a ligand on which an antibody is adsorbed. When the carrier is a particle that is entirely formed of the polymer part, the 2,3-dihydroxypropyl group content in the carrier is preferably 10 micromol/g or more, more preferably 50 micromol/g or more, and most preferably 100 micromol/g or more, based on the solid content in the particle. If the 2,3-dihydroxypropyl group content is less than 10 micromol/g, it may be difficult to obtain a sufficient function of a ligand on which an antibody is adsorbed.

The 2,3-dihydroxypropyl group content can be determined in accordance with JIS K 0070 (determination of the hydroxyl group content by titration). The number of hydroxyl groups is normally twice the number of 2,3-dihydroxypropyl groups.

The purification properties of the carrier according to one embodiment of the invention are not impaired even if a residue derived from a monomer (see “1.2. Production”) is present together with a 2,3-dihydroxypropyl group within the copolymerization range described later. An amino group, a monosaccharide, and a sugar chain polymer such as heparin can also be introduced into the carrier without impairing the antibody purification properties of the carrier. However, introduction of a carboxyl group may impair the antibody purification properties of the carrier. Therefore, the carboxyl group content in the carrier is preferably 10 micromol/g or less, more preferably 1 micromol/g or less, and most preferably 0.1 micromol/g or less, based on the solid content in the particle.

The number average particle size (hereinafter may be referred to as “particle size”) of the carrier according to one embodiment of the invention is preferably 0.1 to 500 micrometers, more preferably 0.3 to 200 micrometers, and most preferably 0.5 to 100 micrometers. The particle size may be determined using a laser diffraction/scattering method.

### 1.2. Production

The carrier according to one embodiment of the invention may be produced by copolymerizing the monomer part to form the polymer part, at least the surface of the carrier being formed of the polymer part. Each monomer included in the monomer part is described below.

### 1.2.1. Monomer (A)

When producing the carrier according to one embodiment of the invention by copolymerizing the monomer part including a monomer (A) that produces a 2,3-dihydroxypropyl group through hydrolysis (hereinafter may be referred to as “monomer (A)”), hydrolysis of the monomer (A) before and during polymerization may be prevented. Note that the monomers (A) to (C) (see below) are preferably a radically polymerizable monomer.

The carrier according to one embodiment of the invention is preferably obtained by hydrolyzing a particle, at least the surface of the particle being formed of the polymer part obtained by copolymerizing the monomer part including the monomer (A). A larger amount of 2,3-dihydroxypropyl group can be stably introduced into the polymer part, and the polymerization stability can be improved by utilizing the monomer (A). It is preferable to remove a residual hydrolysis catalyst from the carrier dispersion after hydrolysis by repeated washing with water using a centrifuge method, a magnetic separation method, or the like.

Examples of the monomer (A) include monomers in which a hydroxyl group is protected by a known protecting group. Specific examples of the monomer (A) include (A-1) a monomer that includes a 2,3-epoxypropyl group, (A-2) a monomer in which a 2,3-dihydroxypropyl group is acetalated, (A-3) a monomer in which a 2,3-dihydroxypropyl group is silylated, and the like.

Specific examples of the monomer (A-1) that includes a 2,3-epoxypropyl group include glycidyl (meth)acrylate, allyl glycidyl ether, and the like. Specific examples of the monomer (A-2) in which a 2,3-dihydroxypropyl group is acetalated include 1,3-dioxolan-2-on-4-ylmethyl (meth)acrylate, 1,3-dioxolane-2,2-dimethyl-4-ylmethyl (meth)acrylate, and the like. Specific examples of the monomer (A-3) in which a 2,3-dihydroxypropyl group is silylated include a di(t-butyl)silylated product of 2,3-dihydroxypropyl (meth)acrylate, a di(trimethylsilylated) product of 2,3-dihydroxypropyl (meth)acrylate, and the like.

The functional groups derived from the monomer (A) are hydrolyzed under appropriate conditions taking account of the type of the monomer (A). The functional groups derived from the monomer (A) are normally hydrolyzed by heating and stirring a dispersion in which the particles are dispersed in water, for several to several tens of hours using an acid, a base, or a fluoride salt as a catalyst. Note that all of the functional groups derived from the monomer (A) included in the copolymer need not necessarily be hydrolyzed as long as the storage stability and the like are not impaired.

The functional groups derived from the monomer (A) are normally hydrolyzed the hydrolyzed after polymerizing the monomer part. Note that some of the functional groups derived from the monomer (A) may be hydrolyzed during polymerization.

The monomer (A) is preferably used in a ratio of 40 to 95 wt %, and more preferably 50 to 90 wt %, based on the monomer part (=100 wt %). If the ratio of the monomer (A) is less than 40 wt %, non-specific adsorption may occur to a large extent. If the ratio of the monomer (A) exceeds 95 wt %, the carrier may be water-soluble, and may not maintain its structure.

### 1.2.2. Monomer (B)

The carrier according to one embodiment of the invention preferably has a surface (particle surface) obtained by copolymerizing a crosslinkable monomer (B) (hereinafter may be referred to as “monomer (B)”). In this case, the monomer part further includes the crosslinkable monomer (B). A porous carrier can be obtained by polymerizing the monomer part including the monomer (B).

The crosslinkable monomer (B) is a monomer that is copolymerizable with the monomer (A) and the like, and includes two or more radically polymerizable unsaturated bonds in one molecule. Examples of the crosslinkable monomer (B) include polyfunctional (meth)acrylates such as ethylene glycol diacrylate, ethylene glycol dimethacrylate, trimethylolpropane triacrylate, trimethylolpropane trimethacrylate, pentaerythritol triacrylate, pentaerythritol trimethacrylate, dipentaerythritol hexaacrylate, and dipentaerythritol hexamethacrylate, conjugated diolefins such as butadiene and isoprene, divinylbenzene, diallyl phthalate, allyl acrylate, allyl methacrylate, and the like. Further examples of the crosslinkable monomer (B) include hydrophilic monomers such as polyethylene glycol diacrylate, polyethylene glycol dimethacrylate, and poly(meth)acrylates of polyvinyl alcohol. The content of the crosslinkable monomer (B) in the copolymer (=100 wt %) is preferably 0 to 30 wt %, and more preferably 5 to 20 wt %. If the content of the monomer (B) in the copolymer exceeds 30 wt %, non-specific adsorption may occur to a large extent.

### 1.2.3. Additional Monomer (C)

The carrier according to one embodiment of the invention may have a surface (particle surface) obtained by copolymerizing an additional monomer (C) other than the monomer (A) and the monomer (B). Examples of the additional monomer (C) include (meth)acrylates including a hydrophilic functional group, such as 2-hydroxyethyl acrylate, 2-hydroxyethyl methacrylate, methoxyethyl acrylate, methoxyethyl methacrylate, polyethylene glycol monoacrylate, and polyethylene glycol monomethacrylate, hydrophilic monomers such as acrylamide, methacrylamide, N-methylolacrylamide, N-methylolmethacrylamide, and diacetoneacrylamide, aromatic vinyl monomers such as styrene, alpha-methylstyrene, and halogenated styrene, vinyl esters such as vinyl acetate and vinyl propionate, unsaturated nitriles such as acrylonitrile, and ethylenically unsaturated alkyl carboxylates such as methyl acrylate, methyl methacrylate, ethyl acrylate, ethyl methacrylate, butyl acrylate, butyl methacrylate, 2-ethylhexyl acrylate, 2-ethylhexyl methacrylate, lauryl acrylate, lauryl methacrylate, stearyl acrylate, stearyl methacrylate, cyclohexyl acrylate, cyclohexyl methacrylate, isobornyl acrylate, and isobornyl methacrylate. A monomer that includes an unprotected 2,3-dihydroxypropyl group, such as 2,3-dihydroxypropyl (meth)acrylate, may be used as the additional monomer (C) as long as the advantageous effects of the invention are not impaired. The additional monomer (C) is used in an amount determined by subtracting the amount of the monomer (A) and the amount of the monomer (B) from the total amount of the monomer part.

### 1.2.4. Polymerization Method

The carrier according to one embodiment of the invention may be produced using a normal method such as emulsion polymerization, soap-free polymerization, or suspension polymerization. For example, the carrier according to one embodiment of the invention may be produced by subjecting the vinyl-based monomers to suspension polymerization or soap-free polymerization. For example, the carrier according to one embodiment of the invention may be produced using the two-step swelling polymerization method disclosed in JP-B-57-24369 that utilizes seed particles (mother particles), the polymerization method described in J. Polym. Sci., Polymer Letters Ed., 21, 937 (1963), or the method disclosed in JP-A-61-215602, JP-A-61-215603, or JP-A-61-215604. Among these, the two-step swelling polymerization method that utilizes seed particles (mother particles) is preferable since the coefficient of variation in particle size can be reduced. Polystyrene or a styrene-based copolymer may be used as the seed particles (mother particles). The polymer part obtained by the two-step swelling polymerization method is a copolymer of the monomers (A) to (C).

Examples of an emulsifier that may be used when copolymerizing the monomers (A) to (C) include anionic surfactants such as alkyl sulfate salts, alkylaryl sulfate salts, alkyl phosphate salts, and fatty acid salts; cationic surfactants such as alkyl amine salts and alkyl quaternary amine salts; nonionic surfactants such as polyoxyethylene alkyl ethers, polyoxyethylene alkylaryl ethers, and block polyethers; amphoteric surfactants such as carboxylic acid-type surfactants (e.g., amino acid-type surfactants and betaine-type surfactants) and sulfonic acid-type surfactants; reactive emulsifiers such as Latemul S-180A, Latemul PD-104 (manufactured by KAO Corp.), Eleminol JS-2 (manufactured by Sanyo Chemical Industries, Ltd.), Aqualon HS-10, Aqualon KH-10, Aqualon RN-10, Aqualon RN-20, Aqualon RN-30, Aqualon RN-50 (manufactured by Dai-ichi Kogyo Seiyaku Co., Ltd.), Adeka Reasoap SE-10N, Adeka Reasoap SR-10, Adeka Reasoap NE-20, Adeka Reasoap NE-30, Adeka Reasoap NE-40 (manufactured by ADEKA Corporation), and Antox MS-60 (manufactured by Nippon Nyukazai Co., Ltd.); and the like. It is preferable to use a reactive emulsifier since the dispersibility of the particles is improved. A polymer that includes a hydrophilic group and has a dispersion function may also be used as the emulsifier. Examples of such a polymer include a styrene-maleic acid copolymer, a styrene-acrylic acid copolymer, polyvinyl alcohol, a polyalkylene glycol, sulfonated polyisoprene, a sulfonated hydrogenated styrene-butadiene copolymer, a sulfonated styrene-maleic acid copolymer, a sulfonated styrene-acrylic acid copolymer, and the like. These emulsifiers may be used either alone or in combination. The emulsifier may be used in an arbitrary amount. The emulsifier is normally used in an amount of 0.1 to 50 parts by weight, preferably 0.2 to 20 parts by weight, and more preferably from 0.5 to 5 parts by weight, based on 100 parts by weight of the monomers (A) to (C) in total. If the amount of the emulsifier is less than 0.1 parts by weight, sufficient emulsification may not occur, and a deterioration in stability may occur during radical polymerization. If the amount of the emulsifier exceeds 50 parts by weight, foaming may occur.

Examples of a radical initiator that may be used when copolymerizing the monomers (A) to (C) include persulfates such as potassium persulfate, sodium persulfate, and ammonium persulfate; water-soluble initiators such as hydrogen peroxide, t-butyl hydroperoxide, t-butylperoximaleic acid, succinic peroxide, and 2,2′-azobis[2-N-benzylamidino]propane hydrochloride; oil-soluble initiators such as benzoyl peroxide, cumene hydroperoxide, diisopropyl peroxydicarbonate, cumyl peroxyneodecanoate, cumyl peroxyoctanoate, and azobisisobutyronitrile; redox initiators using a reducing agent such as acidic sodium sulfite, rongalite, or ascorbic acid; and the like. It is preferable to use an oil-soluble initiator that does not show acidity or basicity in water.

### 1.3. Carrier that Includes Magnetic Material and Method for Producing the Same

The carrier according to one embodiment of the invention may be a particle that includes a magnetic material. For example, the carrier according to one embodiment of the invention may be an organic polymer particle that includes a magnetic material (hereinafter referred to as “magnetic material-containing organic polymer particle”). Since the magnetic material-containing organic polymer particle can be separated using a magnet without using a centrifuge or the like, it is possible to easily or automatically separate the particles from a sample.

Examples of the magnetic material-containing organic polymer particle include (I) a particle in which fine magnetic material particles are dispersed in a continuous phase of a polymer part, (II) a particle that includes a core formed of a secondary aggregate of fine magnetic material particles, and a shell formed of a polymer part, (III) a particle that includes a core particle formed of a non-magnetic material such as an organic polymer, a secondary aggregate layer (magnetic material layer) provided on the surface of the core particle and formed of superparamagnetic fine particles, and a polymer part provided on the magnetic material layer, and the like. Among these, it is preferable to use the particle (III) that includes a core particle that includes a secondary aggregate layer formed of fine magnetic material particles (hereinafter referred to as “mother particle”), and a polymer part provided on the mother particle. Note that the polymer part used for the magnetic material-containing organic polymer particle having each structure includes a 2,3-dihydroxypropyl group. The interface between the core particle and the magnetic material layer provided on the core particle, and the interface between the magnetic material layer and the organic polymer layer provided on the magnetic material layer, may be in a state in which the components of these layers are mixed.

It is most preferable that the magnetic material-containing organic polymer particle have a structure in which the polymer part is provided to cover the core particle, and the magnetic material layer that is provided on the surface of the core particle and formed of superparamagnetic fine particles. In this case, the magnetic material-containing organic polymer particle includes a core and a shell, the core including the core particle, and the magnetic material layer that is provided on the surface of the core particle and formed of superparamagnetic fine particles, and the shell including the polymer part. The polymer part is obtained using the above production method. Specifically, the polymer part (crosslinked polymer) is obtained by polymerizing a monomer part that includes 40 to 95 parts by weight of the monomer (A), 0 to 30 parts by weight of the crosslinkable monomer (B), and 0 to 55 parts by weight of the additional monomer (C) to obtain a copolymer, and hydrolyzing the copolymer.

The mother particles in which the magnetic material layer formed of superparamagnetic fine particles is formed on the surface of the core particle, may be produced by dry-blending non-magnetic organic polymer particles and superparamagnetic fine particles, and physically applying a strong external force to the mixture to form composite particles, for example. The strong force may be physically applied by utilizing a mortar, an automatic mortar, a ball mill, a blade pressure powder compression method, a method that utilizes a mechanochemical effect (e.g., mechanofusion method), or a high-speed air stream impact method (e.g., jet mill or hybridizer). It is desirable to apply a strong physical adsorption force in order to efficiently produce firmly bound composite particles. For example, the particles may be stirred in a vessel equipped with a stirring blade preferably at a stirring blade peripheral velocity of 15 m/sec or more, more preferably 30 m/sec or more, and still more preferably 40 to 150 m/sec. If the stirring blade peripheral velocity is lower than 15 m/sec, energy sufficient to adsorb the superparamagnetic fine particles on the surface of the non-magnetic organic polymer particles may not be obtained. The upper limit of the stirring blade peripheral velocity is not particularly limited. The upper limit of the stirring blade peripheral velocity is determined taking account of the type of apparatus, the energy efficiency, and the like. For example, ferrite and/or magnetite fine particles having a particle size of about 5 to 20 nm may preferably be used as the superparamagnetic fine particles.

The polymer part (shell) may be formed by copolymerizing the monomer part that includes 40 to 95 parts by weight of the monomer (A) that produces a 2,3-dihydroxypropyl group through hydrolysis, 0 to 30 parts by weight of the crosslinkable monomer (B), and 0 to 55 parts by weight of the additional monomer (C), in the presence of the mother particles (core). Each monomer component is the same as described above. A more specific polymerization method is disclosed in JP-A-2004-205481, for example. The hydrolysis conditions employed after polymerization are the same as described above. When the magnetic material-containing organic polymer particles are hydrolyzed under strongly acidic conditions, the superparamagnetic fine particles may be dissolved. Therefore, it is preferable hydrolyze the magnetic material-containing organic polymer particles under weakly acidic to basic conditions.

In order to prevent a situation in which the superparamagnetic fine particles are dissolved, a coating layer may be formed on the mother particles (in which the magnetic material layer formed of the superparamagnetic fine particles is formed on the surface of the core particle) using another monomer part that includes 0 to 30 parts by weight of the crosslinkable monomer (B) and 70 to 100 parts by weight of the additional monomer (C), copolymerizing 40 to 95 parts by weight of the monomer (A), 0 to 30 parts by weight of the crosslinkable monomer (B), and 0 to 55 parts by weight of the additional monomer (C) to obtain particles in which a polymer part (shell) is formed on the mother particle (core) provided with the coating layer, and hydrolyzing the particles to form the magnetic material-containing organic polymer particles. When the magnetic material-containing organic polymer particles have a structure in which the superparamagnetic fine particles are coated with the coating layer, the magnetic material-containing organic polymer particles can be hydrolyzed under strongly acidic to strongly basic conditions.

### 1.4. Applications

The carrier according to one embodiment of the invention is used to purify an antibody by utilizing a 2,3-dihydroxypropyl group as an antibody adsorption ligand to which the antibody binds. Examples of a solution including the purification target antibody include a cell culture including a hybridoma, an antibody solution that has been partially purified by ammonium sulfate precipitation, and the like. The carrier according to one embodiment of the invention may also be used for buffer exchange, desalting, removal of an intended component (e.g., preservative, stabilizer, or surfactant), and the like.

The purification target antibody that is purified using the carrier according to one embodiment of the invention is not particularly limited. Five types of immunoglobulin (i.e., IgG, IgA, IgM, IgD, and IgE) may be purified using the carrier according to one embodiment of the invention. The subclass of IgG is not particularly limited. The carrier according to one embodiment of the invention may be applied to both a polyclonal antibody and a monoclonal antibody.

### 2. ANTIBODY PURIFICATION METHOD

An antibody purification method according to one embodiment of the invention includes bringing a solution that includes an antibody and has a pH of 3.0 or more and less than 5.6 into contact with a carrier that includes a 2,3-dihydroxypropyl group, and bringing the carrier that has been brought into contact with the solution, into contact with an eluent having a pH of 5.6 or more and less than 10. More specifically, the antibody purification method according to one embodiment of the invention includes bringing a solution that includes an antibody and has a pH of 3.0 or more and less than 5.6 into contact with a carrier that includes a 2,3-dihydroxypropyl group to adsorb the antibody on the carrier, and bringing the carrier that has been brought into contact with the solution, into contact with an eluent having a pH of 5.6 or more and less than 10 to elute the antibody from the carrier.

If the pH of the solution is less than 3.0, the antibody may be irreversibly modified. If the pH of the solution is 5.6 or more, the antibody may not be sufficiently adsorbed on the carrier. The pH of the solution is preferably 3.5 or more and less than

### 5.6. Examples of the Solution Include an MES Buffer and the Like

If the pH of the eluent is less than 5.6, the antibody may not be sufficiently eluted into the eluent. If the pH of the eluent is 10 or more, it may be difficult to use the eluent directly for subsequent reactions. The pH of the eluent is preferably 6.0 or more and less than 10. Examples of the eluent include a phosphate buffer, a borate buffer, a HEPES buffer, a Tris buffer, and the like.

Examples of the purification target antibody that is purified using the antibody purification method according to one embodiment of the invention include those mentioned above in connection with the section entitled “1.4. Applications”.

### 3. EXAMPLES

The invention is further described below by way of examples. Note that the invention is not limited to the following examples.

### 3.1. Synthesis Example 1

### Synthesis of Porous Particles Including a 2,3-Dihydroxypropyl Group as a Ligand

110 g of glycidyl methacrylate (manufactured by Mitsubishi Rayon Co., Ltd., hereinafter referred to as “GMA”) and 73 g of trimethylolpropane trimethacrylate (manufactured by Sartomer, hereinafter referred to as “TMP”) were dissolved in 200 g of diisobutyl ketone (manufactured by Mitsui Chemicals Inc.) and 97 g of acetophenone (manufactured by Wako Pure Chemical Industries, Ltd.). 2.4 g of 2,2′-azoisobutyronitrile (manufactured by Wako Pure Chemical Industries, Ltd.) was added to the solution to prepare an organic monomer solution.

17 g of polyvinyl alcohol (“PVA-217” manufactured by Kuraray Co., Ltd.), 1.7 g of sodium dodecyl sulfate (“Emal 10G” manufactured by Kao Corporation), and 85 g of sodium chloride were added to 4250 g of purified water. The mixture was stirred overnight to prepare an aqueous solution.

A separable flask (7 L) provided with a baffle was charged with the organic monomer solution and the aqueous solution. The separable flask was equipped with a thermometer, a stirring blade, and a cooling tube, and placed in a hot water bath, and the mixture was stirred at 350 rpm in a nitrogen atmosphere. The separable flask was then heated using the hot water bath, and the mixture was stirred at 85° C. for 5 hours.

After cooling the reaction mixture, the resulting particles were washed with isopropyl alcohol and water. After dispersing the particles using an ultrasonic bath, the fine particles contained in the supernatant liquid were removed by decantation. This operation was repeated three times. The particles were then dried using a vacuum dryer to obtain particles (103 g) including an epoxy group. The modal pore size and the specific surface area of the particles were measured using a mercury porosimeter (“AutoPore IV950” manufactured by Shimadzu Corporation) (pore size measurement range: 0.5 to 5000 nm), and found to be 227 nm and 166 m²/g, respectively. The volume average particle size of the particles measured using a laser diffraction/scattering particle size distribution analyzer (“LS13320” manufactured by Beckman Coulter, Inc.) was 30 micrometers.

10 g of the particles were put in a polyethylene bottle (250 mL). The particles were dispersed in 80 g of purified water, followed by the addition of 10 g of 0.1 M sulfuric acid. The mixture was inversion-mixed at 60° C. for 5 hours to ring-open the epoxy group to obtain porous particles including a 2,3-dihydroxypropyl group as a ligand. The porous particles were repeatedly washed with purified water, and dispersed in purified water so that the solid content was 2 wt %.

The hydroxyl group content in the particles was measured in accordance with JIS K 0070. Specifically, some of the particles were dried under reduced pressure, and 0.2 g of the particles were weighed. After the addition of 5 mL of an acetic anhydride/pyridine solution as an acetylating agent, the mixture was reacted at 100° C. for 1 hour. After allowing the mixture to cool, 1 mL of purified water was added to the mixture. The mixture was heated at 100° C. for 10 minutes to decompose acetic anhydride. After allowing the mixture to cool, titration was performed using a 0.5 M potassium hydroxide ethanol solution (indicator: phenolphthalein) to determine the hydroxyl group content. The hydroxyl group content was found to be 4.9 mmol/g.

### 3.2. Example 1

### Purification of Antibody from Partially Purified IgG Subjected to Ammonium Sulfate Precipitation

A hybridoma that produces a mouse monoclonal IgG antibody was cultured in the presence of 20% fetal bovine serum from which the bovine IgG had been removed. A mouse monoclonal IgG antibody produced in the culture supernatant was precipitated using 50% saturated ammonium sulfate. The precipitate was dissolved in a PBS(−) buffer, and dialyzed to desalt unnecessary ammonium sulfate to obtain a partially purified IgG (immunoglobulin G) solution. The protein concentration in the solution calculated from absorbance measurement was 3.3 mg/mL, on the assumption that the protein concentration when the absorbance at 280 nm is 1.4 is 1.0 mg/mL. The IgG purity determined by SDS-PAGE was 40%. Note that the purified IgG solution may include bovine serum albumin derived from the medium and the like as impurities. The antibody purification method according to one embodiment of the invention was applied to the partially purified IgG solution.

10 microliters of the partially purified IgG solution and 140 microliters of a 100 mM MES buffer (pH: 5.0) were added to 50 microliters of the porous particles obtained in Synthesis Example 1, and the mixture was shaken at room temperature for 60 minutes. After centrifuging the mixture at 10,000 g for 5 minutes, the supernatant liquid including the particles was collected using a pipette (adsorption step).

After the addition of 1 mL of a 100 mM MES buffer (pH: 5.0) to the particles, the particles were dispersed, washed, and centrifuged, and the supernatant liquid was removed using a pipette (washing step). The washing step was repeated twice.

After the addition of 200 microliters of a 100 mM phosphate buffer (pH: 7.4) to the particles, the mixture was shaken at room temperature for 10 minutes to elute IgG adsorbed on the particles (elution step).

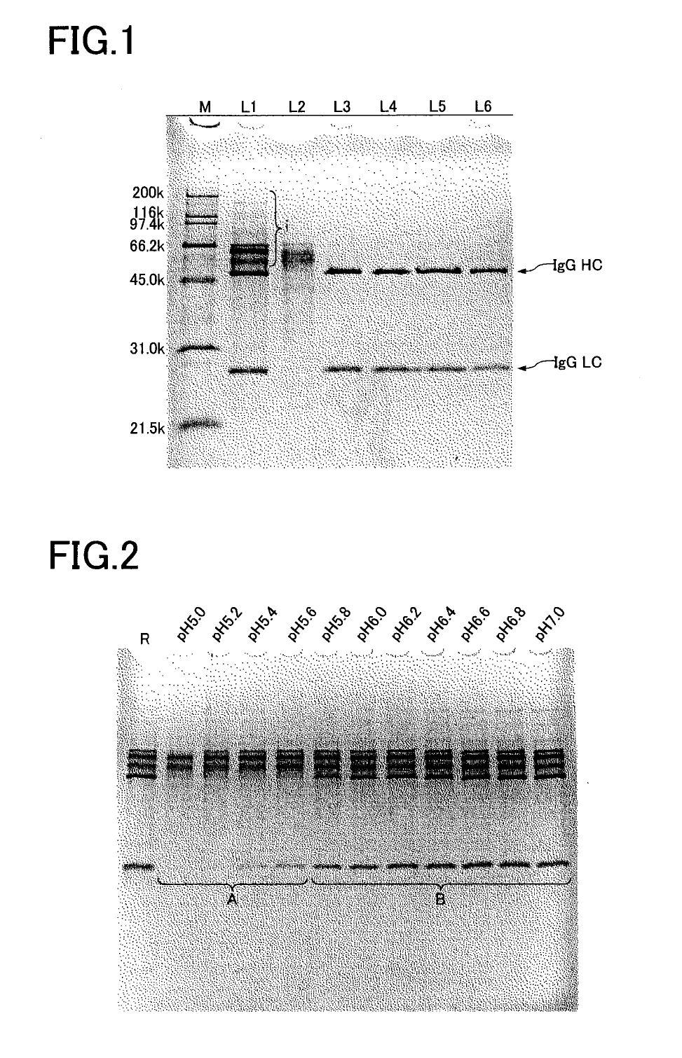

0.5 microliters of the partially purified IgG solution, 10 microliters of the solution obtained by the adsorption step, and 10 microliters of the solution collected in the elution step were assayed by SDS-PAGE (see FIG. 1).

The lanes 1 to 6 (L1 to L6) in FIG. 1 represent the pattern of partially purified IgG, the supernatant liquid obtained by the adsorption step, an eluent obtained using a 100 mM phosphate buffer (pH: 7.4), an eluent obtained using a 100 mM phosphate buffer+0.5 M NaCl (pH: 7.4), an eluent obtained using a 100 mM borate buffer (pH: 8.5), and an eluent obtained using a 100 mM borate buffer+0.5 M NaCl (pH: 8.5). In FIG. 1, M represents a molecular weight marker, i represents impurities, IgG HC represents an IgG heavy chain, and IgG LC represents an IgG light chain.

As illustrated in FIG. 1, impurities included in the partially purified IgG solution (lane 1) did not bind to the particles in the adsorption step, and were observed in the supernatant liquid (lane 2). IgG adsorbed on the particles was eluted in the elution step, and collected in the supernatant liquid of the eluent (lane 3). The purity of the collected IgG was 99% or more, and the collection rate was 95% or more.

In order to determine the effects of the pH and the salt concentration of the eluent, the elution effect was determined using a 100 mM phosphate buffer (pH: 7.4) including 0.5 M sodium chloride (lane 4), a 100 mM borate buffer (pH: 8.5) (lane 5), or a 100 mM borate buffer (pH: 8.5) including 0.5 M sodium chloride (lane 6), instead of a 100 mM phosphate buffer. It was found that IgG could be eluted with high efficiency when using a solution having a high salt concentration (0.5 M) (lanes 4 to 6) as the eluent, and when using a weak alkaline solution having a pH of 8.5 (lanes 5 and 6) as the eluent.

It was thus confirmed that IgG included in the partially purified IgG solution could be collected with high purity as an almost neutral buffer solution using the antibody purification method according to one embodiment of the invention. Since IgG can be collected as an almost neutral buffer solution, IgG can be subjected directly to another step (e.g., determination of IgG).

### 3.3. Example 2

### Effects of pH in Antibody Adsorption Step

1 mg of the porous particles including a 2,3-dihydroxypropyl group as a ligand that was obtained in Synthesis Example 1 were dispersed in 190 microliters of a 100 mM MES buffer of which the pH was adjusted to a value within the range of 5.0 to 7.0 (i.e., the pH was increased from 5.0 to 7.0 at intervals of 0.2 (see FIG. 2)). After the addition of 10 microliters of a partially purified IgG solution, the mixture was shaken at room temperature for 60 minutes. After centrifuging the mixture at 10,000 g for 5 minutes, the supernatant liquid of the dispersion was collected using a pipette (adsorption step).

10 microliters of the supernatant liquid collected in the adsorption step was assayed by SDS-PAGE (see FIG. 2).

As illustrated in FIG. 2, the amount of IgG included in the supernatant liquid decreased when treating the porous particles obtained in Synthesis Example 1 using a buffer having a pH of 5.0 to 5.6. It was thus confirmed that IgG included in the partially purified IgG solution preferentially bound to the particles including a 2,3-dihydroxypropyl group that was obtained in Synthesis Example 1 within a pH range of 5.0 to 5.6. In FIG. 2, R represents partially purified IgG, A represents an adsorption range, and B represents a non-adsorption range.

### 3.4. Comparative Example 1

### Acetylation of Porous Particles Including a 2,3-dihydroxypropyl Group as a Ligand

1.0 g of a dry powder of the porous particles including a 2,3-dihydroxypropyl group as a ligand that was obtained in Synthesis Example 1 was dispersed in 9.0 g of pyridine to prepare a porous particle dispersion.

Pyridine was added to 2.5 g of acetic anhydride to prepare 10 mL of a solution. After the addition of 1.0 mL of the solution to the porous particle dispersion, the mixture was reacted at 100° C. for 2 hours. After the addition of 2.0 mL of purified water, the mixture was reacted at 100° C. for 1 hour. The particles were collected using a Kiriyama funnel, sufficiently washed with purified water, and dried to obtain acetylated particles in which the 2,3-dihydroxypropyl group was acetylated.

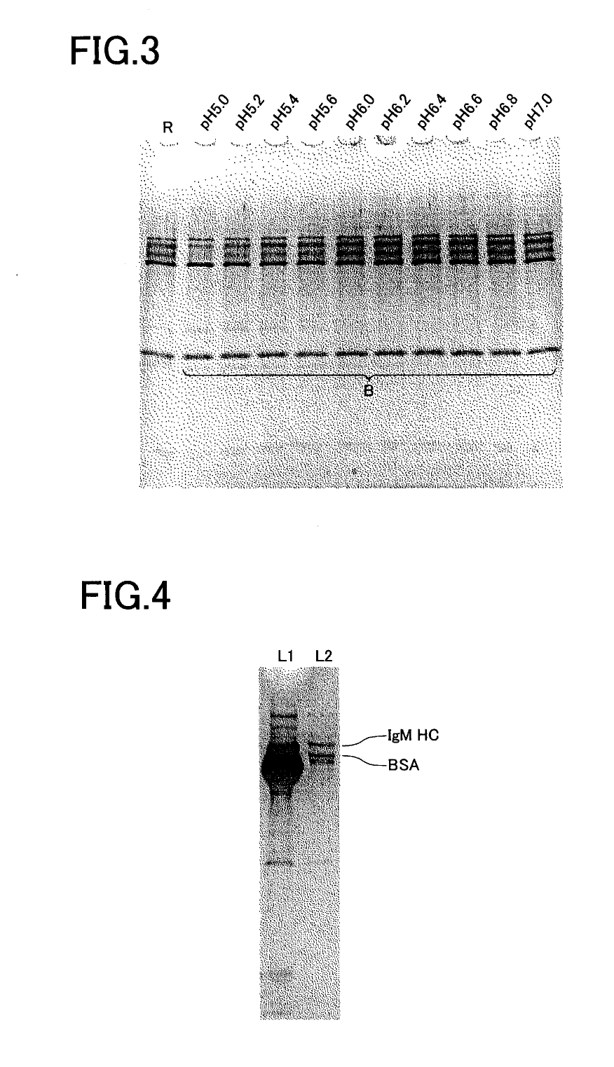

1 mg of the acetylated particles were dispersed in 190 microliters of a 100 mM MES buffer of which the pH was adjusted to a value within the range of 5.0 to 7.0 (i.e., the pH was increased from 5.0 to 7.0 at intervals of 0.2 (see FIG. 3)). After the addition of 10 microliters of a partially purified IgG solution, the mixture was shaken at room temperature for 60 minutes. After centrifuging the mixture at 10,000 g for 5 minutes, the supernatant liquid was collected using a pipette (adsorption step).

10 microliters of the supernatant liquid collected in the adsorption step was assayed by SDS-PAGE (see FIG. 3).

As illustrated in FIG. 3, IgG included in the partially purified IgG solution was not adsorbed on the acetylated particles. Specifically, impurities and IgG could not be separated when using the acetylated particles. It was thus confirmed that purification of IgG achieved in Example 1 was due to specific binding of the 2,3-dihydroxypropyl group and IgG. In FIG. 3, R represents partially purified IgG, and B represents a non-adsorption range.

### 3.5. Example 3

### Purification of IgM Antibody from Hybridoma Culture Supernatant

A hybridoma that produces a mouse monoclonal IgM antibody was cultured in the presence of 20% fetal bovine serum. The IgM concentration in the culture supernatant was determined by ELISA, and found to be 30 micrograms/mL. The IgM content with respect to the total proteins was 0.2%. IgM was purified directly from the hybridoma culture supernatant using the particles obtained in Synthesis Example 1.

100 microliters of the culture supernatant and 500 microliters of a100 mM MES buffer (pH: 5.0) were added to 50 microliters of the porous particles obtained in Synthesis Example 1, and the mixture was shaken at room temperature for 60 minutes. After centrifuging the mixture at 10,000 g for 5 minutes, the supernatant liquid including the particles was collected using a pipette (adsorption step).

After the addition of 1 mL of a 100 mM MES buffer (pH: 5.0) to the particles, the particles were dispersed, washed, and centrifuged, and the supernatant liquid was removed using a pipette (washing step). The washing step was repeated twice.

After the addition of 60 microliters of a 100 mM phosphate buffer (pH: 7.4) to the particles, the mixture was shaken at room temperature for 10 minutes to elute IgM adsorbed on the particles (elution step).

4.5 microliters of the solution collected in the elution step (corresponding to 7.5 microliters of the culture) was assayed by reducing SDS-PAGE (see FIG. 4).

The lane 1 (L1) and the lane 2 (L2) in FIG. 4 represent the pattern of the culture supernatant (2.5 micrograms/lane) and an eluent obtained using a phosphate buffer (pH: 7.4). In FIG. 4, IgM HC represents an IgM heavy chain.

As illustrated in FIG. 4, a large amount of BSA derived from the bovine serum was observed in the culture supernatant (lane 1). On the other hand, most of the proteins derived from the bovine serum were removed from the supernatant liquid of the eluent obtained by purification (lane 2), and a band attributed to IgM was clearly observed. The purity of the collected IgM was 20% or more although the culture supernatant having a low IgM concentration was used as the starting material. The collection rate was 90% or more.

### 3.6. Synthesis Example 2

### (Synthesis of Organic Polymer Particles Including a 2,3-dihydroxypropyl Group on the Surface Thereof as a Ligand, and Including a Magnetic Material Inside the Organic Polymer Particles)

2 parts by mass of a di(3,5,5-trimethylhexanoyl) peroxide solution (“Peroyl 355-75(S)” manufactured by NOF Corporation) and 20 parts by mass of a 1% sodium dodecylsulfate aqueous solution were mixed, and finely emulsified using an ultrasonic disperser. The resulting emulsion was added to a reactor charged with 13 parts by mass of polystyrene particles having a particle size of 0.77 micrometers and 41 parts by mass of water, and the mixture was stirred at 25° C. for 12 hours. 96 parts by mass of styrene and 4 parts by mass of divinylbenzene were emulsified in another vessel using 400 parts by mass of a 0.1% sodium dodecyl sulfate aqueous solution. The resulting emulsion was added to the reactor. After stirring the mixture at 40° C. for 2 hours, the mixture was heated to 75° C., and polymerized for 8 hours. After cooling the mixture to room temperature, the resulting particles were separated by centrifugation, washed with water, dried, and ground. The ground particles were used as core particles (preparation of core particles). The number average particle size of the core particles was 1.5 micrometers.

Acetone was added to an oily magnetic fluid (“EXP series” manufactured by Ferrotec Corporation) to precipitate particles, and the particles were dried to obtain ferrite-based fine magnetic material particles (average primary particle size: 0.01 micrometers) having a hydrophobized surface.

15 g of the core particles and 15 g of the hydrophobized fine magnetic material particles were thoroughly mixed using a mixer. The mixture was treated using a hybridization system (“NHS-0” manufactured by Nara Machinery Co., Ltd.) at a blade (stirring blade) peripheral velocity of 100 msec (16,200 rpm) for 5 minutes to obtain mother particles having a surface magnetic material layer formed of fine magnetic material particles having a number average particle size of 2.0 micrometers.

A separable flask (1 L) was charged with 375 g of an aqueous solution (hereinafter referred to as “polymerization solvent”) including 0.25 wt % of sodium dodecylbenzenesulfonate and 0.25 wt % of a nonionic emulsifier (“Emulgen 150” manufactured by Kao Corporation). 15 g of the mother particles having the magnetic material layer were added to the flask, and dispersed using a homogenizer, and the resulting dispersion was heated to 60° C. A pre-emulsion prepared by dispersing 27 g of methyl methacrylate (hereinafter referred to as “MMA”), 3 g of TMP, and 0.6 g of Peroyl 355-75(S) in 150 g of the polymerization solvent, was added dropwise to the separable flask controlled at 60° C. over 1 hour and 30 minutes. After the dropwise addition, the mixture was stirred at 60° C. for 1 hour. A pre-emulsion prepared by dispersing 13.5 g of GMA, 1.5 g of TMP, and 0.3 g of Peroyl 355-75(S) in 75 g of the polymerization solvent, was added dropwise to the separable flask controlled at 60° C. over 1 hour and 30 minutes. After heating the mixture to 75° C., the mixture was polymerized for 2 hours to complete the reaction. After the addition of 60 mL of 1 mol/L sulfuric acid to the separable flask, the mixture was stirred at 60° C. for 6 hours. The particles contained in the separable flask were separated magnetically, and repeatedly washed with distilled water. Organic polymer particles including a 2,3-dihydroxypropyl group on the surface thereof, and including a magnetic material were thus obtained. The particles were dispersed in purified water so that the solid content was 10 wt %.

The hydroxyl group content in the particles was measured in the same manner as in Synthesis Example 1, and found to be 0.45 mmol/g.

### 3.7. Example 4

### Antibody Purification Using Organic Polymer Particles Including a 2,3-dihydroxypropyl Group on the Surface Thereof as a Ligand, and Including a Magnetic Material Inside the Organic Polymer Particles

A test tube was charged with 10 microliters of the magnetic particles obtained in Synthesis Example 2, 1.5 microliters of a partially purified IgG solution, and 38.5 microliters of a 100 mM MES buffer (pH: 5.0). The mixture was shaken at room temperature for 10 minutes. The purity of IgG included in the partially purified IgG solution determined by SDS-PAGE was 38%. Bovine serum albumin derived from the medium and the like were included as impurities. The tube was placed on a magnetic stand to magnetically separate the particles, and the supernatant liquid including the particles was collected using a pipette (adsorption step).

After the addition of 50 microliters of a 100 mM MES buffer (pH: 5.0) to the particles, the particles were dispersed, and washed. The tube was placed on a magnetic stand to magnetically separate the particles, and the supernatant liquid was collected using a pipette (washing step). The washing step was repeated twice.

After the addition of 50 microliters of a 100 mM phosphate buffer (pH: 7.4) to the particles, the mixture was shaken at room temperature for 10 minutes to elute IgG adsorbed on the particles (elution step).

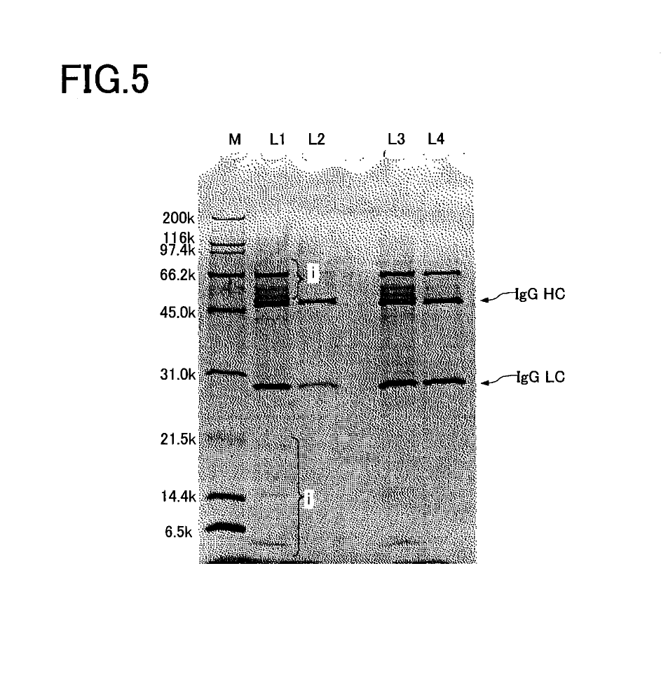

0.18 microliters of the partially purified IgG solution, and 6 microliters of the solution collected in the elution step were assayed by reducing SDS-PAGE (see FIG. 5).

The lane 1 (L1) and the lane 2 (L2) in FIG. 5 represent the pattern of the partially purified IgG solution and an eluent obtained using a phosphate buffer (pH: 7.4) (Example 4). In FIG. 5, M represents a molecular weight marker, i represents impurities, IgG HC represents an IgG heavy chain, and IgG LC represents an IgG light chain.

As illustrated in FIG. 5, impurities included in the partially purified IgG solution (lane 1) were observed to only a small extent in the supernatant liquid (IgG solution) of the eluent (lane 2). The purity of the collected IgG was 98%, and the collection rate was 90%.

It was thus confirmed that IgG could be efficiently and easily purified from the partially purified IgG solution by utilizing the organic polymer particles including a 2,3-dihydroxypropyl group on the surface thereof as a ligand, and including a magnetic material inside the organic polymer particles.

### 3.8. Synthesis Example 3

### Synthesis of Organic Polymer Particles Including a Carboxyl Group and a 2,3-dihydroxypropyl Group on the Surface Thereof as a Ligand, and Including a Magnetic Material Inside the Organic Polymer Particles

A separable flask (1 L) was charged with 375 g of the polymerization solvent. 15 g of the mother particles (having the surface magnetic material layer formed of fine magnetic material particles having a number average particle size of 2.0 micrometers) synthesized in Synthesis Example 2 were added to the flask, and dispersed using a homogenizer, and the resulting dispersion was heated to 60° C. A pre-emulsion prepared by dispersing 27 g of MMA, 3 g of TMP, and 0.6 g of Peroyl 355-75(S) in 150 g of the polymerization solvent, was added dropwise to the separable flask controlled at 60° C. over 1 hour and 30 minutes. After the dropwise addition, the mixture was stirred at 60° C. for 1 hour. A pre-emulsion prepared by dispersing 3 g of t-butyl methacrylate, 10.5 g of GMA, 1.5 g of TMP, and 0.3 g of Peroyl 355-75(S) in 75 g of the polymerization solvent, was added dropwise to the separable flask controlled at 60° C. over 1 hour and 30 minutes. After heating the mixture to 75° C., the mixture was polymerized for 2 hours to complete the reaction. A copolymer covering the mother particles (core) was thus formed. 60 mL of 1 mol/L sulfuric acid was added to the separable flask, and the mixture was stirred at 60° C. for 6 hours to effect a hydrolysis reaction to obtain organic polymer particles (organic polymer particles exhibiting low non-specific adsorption) including the mother particle as a core, and including a polymer part as a shell. The particles contained in the separable flask were separated magnetically, and repeatedly washed with distilled water. The particles were dispersed in purified water so that the solid content was 10 wt %. A dispersion of the organic polymer particles including a magnetic material was thus obtained.

The particle size of the particles was 2.8 micrometers, and the carboxyl group content determined by conductometric titration was 24 micromol/g.

### 3.9. Reference Example 1

### Antibody Purification Using Organic Polymer Particles Including a Carboxyl Group and a 2,3-dihydroxypropyl Group on the Surface Thereof as a Ligand, and Including a Magnetic Material Inside the Organic Polymer Particles

A test tube was charged with 10 microliters of the magnetic particles obtained in Synthesis Example 3, 1.5 microliters of the partially purified IgG solution used in Example 4, and 38.5 microliters of a 100 mM MES buffer (pH: 5.0). The mixture was shaken at room temperature for 10 minutes. The tube was placed on a magnetic stand to magnetically separate the particles, and the supernatant liquid including the particles was collected using a pipette (adsorption step).

After the addition of 50 microliters of a 100 mM MES buffer (pH: 5.0) to the particles, the particles were dispersed, and washed. The tube was placed on a magnetic stand to magnetically separate the particles, and the supernatant liquid was collected using a pipette (washing step). The washing step was repeated twice.

After the addition of 50 microliters of a 100 mM phosphate buffer (pH: 7.4) to the particles, the mixture was shaken at room temperature for 10 minutes to elute IgG adsorbed on the particles (elution step).

0.18 microliters of the partially purified IgG solution, and 6 microliters of the solution collected in the elution step were assayed by reducing SDS-PAGE (see FIG. 5).

The lane 3 (L3) and the lane 4 (L4) in FIG. 5 represent the pattern of the partially purified IgG solution and an eluent obtained using a phosphate buffer (pH: 7.4) (Reference Example 1). In FIG. 5, M represents a molecular weight marker, i represents impurities, IgG HC represents an IgG heavy chain, and IgG LC represents an IgG light chain.

As illustrated in FIG. 5, while impurities included in the partially purified IgG solution (lanes 1 and 3) were observed to only a small extent when using the particles of Example 4 (lane 2), most of the impurities remained in the solution collected in the elution step when using the particles of Reference Example 1 (i.e., sufficient purification could not be achieved) (lane 4). The purity of the collected IgG was 58%, and the collection rate was 95%.

The invention is not limited to the above embodiments. Various modifications and variations may be made of the above embodiments. For example, the invention includes various other configurations substantially the same as the configurations described in connection with the above embodiments (e.g., a configuration having the same function, method, and results, or a configuration having the same objective and results). The invention also includes a configuration in which an unsubstantial section (element) described in connection with the above embodiments is replaced with another section (element). The invention also includes a configuration having the same effects as those of the configurations described in connection with the above embodiments, or a configuration capable of achieving the same objective as that of the configurations described in connection with the above embodiments. The invention further includes a configuration in which a known technique is added to the configurations described in connection with the above embodiments.

### REFERENCE SIGNS LIST

M: molecular weight marker, L1: lane 1, L2: lane 2, L3: lane 3, L4: lane 4, L5: lane 5, L6: lane 6, i: impurities, IgG HC: IgGheavy chain, IgG LC: IgG light chain, R: partially purified IgG, A: adsorption range, B: non-adsorption range, IgM HC: IgM heavy chain, BSA: bovine serum albumin

1. An antibody purification method comprising:
contacting a solution comprising an antibody and having a pH of 3.0 or more and less than 5.6 with a carrier comprising a 2,3-dihydroxypropyl group; and then
contacting the carrier with an eluent having a pH of 5.6 or more and less than 10.

2. An antibody purification carrier comprising a 2,3-dihydroxypropyl group, wherein the 2,3-dihydroxypropyl group is present on a surface of the antibody purification carrier.

3. The antibody purification carrier according to claim 2, wherein the antibody purification carrier comprises a porous particle.

4. The antibody purification carrier according to claim 2, wherein the antibody purification carrier comprises a particle comprising a magnetic material.

5. The antibody purification carrier according to claim 3, wherein the antibody purification carrier comprises a particle comprising a magnetic material.

6. The antibody purification method according to claim 1, wherein the solution has a pH of 3.5 or more and less than 5.6.

7. The antibody purification method according to claim 1, wherein the eluent has a pH of 6.0 or more and less than 10.

8. The antibody purification method according to claim 1, wherein the solution has a pH of 3.5 or more and less than 5.6, and the eluent has a pH of 6.0 or more and less than 10.
